(19) United States
(12) Patent Application Publication
(10) Pub. No.: US 2006/0287686 A1
(43) Pub. Date: Dec. 21, 2006

(54) System and method for implantable device with one or more stored treatment protocols and transmission to external device

(75) Inventors: Patrick M. Cullen, Dallas, TX (US); John H. Erickson, Plano, TX (US); Galen L. Smith, Allen, TX (US)

Correspondence Address: FULBRIGHT & JAWORSKI, L.L.P. (ANS), 2200 ROSS AVENUE, SUITE 2800, DALLAS, TX, 75201-2784, US

(73) Assignee: Advanced Neuromodulation Systems, Inc., Plano, TX (US)

(21) Appl. No.: 11/029,662

(22) Filed: Jan. 5, 2005

Related U.S. Application Data
(63) Continuation-in-part of application No. 10/613,142, filed on Jul. 3, 2003.

(51) Int. Cl. A61N 1/362

(52) U.S. Cl. 607/30

(57) ABSTRACT

A system, method, and implantable pulse generator (IPG) device that stores, on the implantable device, two or more treatment-protocol stimulus programs, each preferably having one or more stimsets, preferably as prescribed by a physician. The IPG communicates with an external patient programmer (EPP) to determine which of the stimulus programs should be run at any given time. Programs stored on the IPG are uploaded to the external device, and a selected program retransmitted (either the program itself, or selection information identifying the selected one of the IPG-stored programs) to the IPG for execution. An advanced programmer is used to read and write program instructions to the IPG. In this way, the patient is capable of carrying two or more program options with him, and if the patient uses an EPIPG, he can use any available EPP to power and operate the EPIPG.

### CROSS-REFERENCE TO RELATED APPLICATION

The present application is a continuation-in-part of U.S. patent application Ser. No. 10/613,142, filed Jul. 3, 2003, for “System And Method for Implantable Pulse Generator with Multiple Treatment Protocols” which is incorporated herein by reference.

### TECHNICAL FIELD OF THE INVENTION

The present invention is directed, in general, to medical devices and, more specifically, to implantable stimulation or sensing devices (i.e. active implant).

### BACKGROUND

The present invention relates to stimulation systems, for example, spinal cord, peripheral, and deep-brain stimulation systems. A spinal cord stimulation system is an implantable pulse generating system used to provide electrical stimulation pulses from an electrode array placed epidurally or surgically near a patient's spine. An implanted pulse generator (IPG) may operate independently to provide the required electrical stimulation, or may interact with an external programmer, which delivers programming and/or control information and/or energy for the electrical stimulation, typically through a radio-frequency (RF) or other wireless signal.

Spinal cord stimulation (SCS) is a well accepted clinical method for reducing pain in certain populations of patients. SCS systems typically include an implanted device, lead wires, and electrodes connected to the lead wires. The implanted device receives signals from an external programmer, and transmits corresponding electrical pulses that are delivered to the spinal cord (or other tissue) through the electrodes which are implanted along the dura of the spinal cord. In a typical situation, the attached lead wires exit the epidural space and are tunneled around the torso of the patient to a sub-cutaneous pocket where the device is implanted.

Spinal cord and other stimulation systems are known in the art. For example, in U.S. Pat. No. 3,646,940, there is disclosed an implantable electronic stimulator that provides timed sequenced electrical impulses to a plurality of electrodes so that only one electrode has a voltage applied to it at any given time. Thus, the electrical stimuli provided by the apparatus taught in the '940 patent comprise sequential, or non-overlapping, stimuli.

In U.S. Pat. No. 3,724,467, an electrode implant is disclosed for the neurostimulation of the spinal cord. A relatively thin and flexible strip of physiologically inert plastic is provided with a plurality of electrodes formed thereon. The electrodes are connected by leads to an RF receiver, which is also implanted, and which is controlled by an external controller. The implanted RF receiver has no power storage means for generating electrical stimulations, and must be coupled to the external controller in order for neurostimulation to occur.

In U.S. Pat. No. 3,822,708, another type of electrical spinal cord stimulating device is shown. The device has five aligned electrodes which are positioned longitudinally on the spinal cord and transversely to the nerves entering the spinal cord. Current pulses applied to the electrodes are said to block sensed intractable pain, while allowing passage of other sensations. The stimulation pulses applied to the electrodes are approximately 250 microseconds in width with a repetition rate of 5 to 200 pulses per second. A patient-operable switch allows the patient to change which electrodes are activated, i.e., which electrodes receive the current stimulus, so that the area between the activated electrodes on the spinal cord can be adjusted, as required, to better block the pain.

Other representative patents that show spinal cord stimulation systems or electrodes include U.S. Pat. Nos. 4,338,945; 4,379,462; 5,121,754; 5,417,719, 5,501,703, and 6,516,227. All of the patents noted above are hereby incorporated by reference.

A typical IPG is self contained, having a multi-year battery pack and a single treatment program, and is generally programmed during or immediately following implantation in the patient's body.

Other SCS systems have no implanted power source, but receive power and programming and/or control information from an external transmitter. These systems will convert the RF signals from the transmitter to provide power to the implanted receiver, and use the RF programming information to determine the intensity, location, and duration of the electrical pulses delivered to the electrodes.

There is a significant programming limitation with known SCS systems. In a typical IPG, the patient's program is installed during implantation, and the patient must visit a doctor to have any programming changes made.

In an externally-powered SCS system, or in an internally-powered SCS system wherein only a single program is communicated from the external programmer and stored within a control register of the IPG (which controls the operation of the IPG) such as described in U.S. Pat. No. 6,381,496, which is incorporated herein by reference, the external programmer/transmitter carries the patient's programming of which a single program is communicated to the implanted receiver. In order to prevent mistaken use of another, differently-programmed programmer/transmitter, the patient's transmitter is effectively “tied” to the patient's receiver for the entire life of the receiver. If the patient should use another programmer/transmitter, it will send the receiver a stimulation program that may be inappropriate or even harmful to the patient.

In such applications, since all programming for an SCS receiver is stored on the transmitter, the patient must carry that specific transmitter with him whenever he requires a change in prescription or programming, since the transmitter itself must be reprogrammed.

There is, therefore, a need in the art for a system, process and device for improved programming options for IPGs.

### SUMMARY

In one embodiment of the present invention, there is a provided an implantable device including a processor operable for controlling operation of the implantable device and a memory coupled to the processor and operable for storing a treatment program. A communication circuit is operable for transmitting one or more stored treatment programs to an external device. In a further embodiment, once uploaded from the implantable device, a program is selected, validated and retransmitted to the IPG for execution.

In another embodiment, there is provided a method of controlling operation of an implantable device. The method includes storing within a memory in the implantable device a treatment program, where the treatment program is operable for controlling the implantable device. The stored treatment program is transmitted from the implantable device to the external device. In a further embodiment, memory within the external device is cleared of previously stored treatment programs.

In yet another embodiment, there is provided an external device (and means) operable for monitoring one or more treatment programs stored within an implantable device. The external device includes a communication circuit operable for wirelessly receiving from the implantable device the one or more treatment programs stored within the implantable device, a display means operable for displaying information related to the one or more treatment programs to a user, and an input means operable for selecting one of the one or more of the received treatment programs to control operation of the implantable device. The communication circuit is further operable for transmitting the selected treatment program to the implantable device, and the transmitted selected treatment program is operable for controlling operation of the implantable device. In a further embodiment, the external device includes memory for storing treatment programs and means for clearing the memory, the memory operable for storing the one or more received treatment programs from the implantable device.

In still another embodiment, there is provided a medical system having an implantable device and an external device. The implantable device is operable for implantation into a body, and further includes a memory operable for storing one or more treatment programs, the one or more treatment programs operable for controlling operation of the implantable device, and a communication circuit operable, when the implantable device is implanted within the body, for transmitting the stored one or more treatment programs. The external device is operable for receiving the one or more treatment programs transmitted from the implantable device operable for monitoring one or more treatment programs stored within an implantable device. The external device includes a communication circuit operable for wirelessly receiving from the implantable device the one or more treatment programs stored within the implantable device, a display means operable for displaying information related to the one or more treatment programs to a user, and an input means operable for selecting one of the one or more of the received treatment programs to control operation of the implantable device. The communication circuit is further operable for transmitting the selected treatment program to the implantable device, and the transmitted selected treatment program is operable for controlling operation of the implantable device.

In another embodiment, there is provided a method of controlling the operation of an implanted device in a medical system. The method includes storing one or more treatment programs within a memory in the implanted device, where the one or more treatment programs each operable for controlling the implantable device. The one or more stored treatment programs are transmitted from the implantable device and received at the external device. One of the treatment program(s) received by the external device is selected and the selected treatment program is transmitted from the external device and received at the implanted device. The operation of the implantable device is controlled in response to the received treatment program. In a further embodiment, memory within the external device is cleared of previously stored treatment programs.

The foregoing has outlined rather broadly the features and technical advantages of the present invention so that those skilled in the art may better understand the detailed description of the invention that follows. Additional features and advantages of the invention will be described hereinafter that form the subject of the claims of the invention. Those skilled in the art will appreciate that they may readily use the conception and the specific embodiment disclosed as a basis for modifying or designing other structures for carrying out the same purposes of the present invention. Those skilled in the art will also realize that such equivalent constructions do not depart from the spirit and scope of the invention in its broadest form.

### BRIEF DESCRIPTION OF THE DRAWINGS

For a more complete understanding of the present invention, and the advantages thereof, reference is now made to the following descriptions taken in conjunction with the accompanying drawings, wherein like numbers designate like objects, and in which:

FIG. 1 depicts a block diagram of an implantable pulse generator in accordance with a preferred embodiment of the present invention;

FIG. 2 depicts a flowchart of a process in accordance with an embodiment of the invention;

FIG. 3 depicts a flowchart of a process in accordance with an embodiment of the invention;

FIG. 4A illustrates the general operation of one embodiment of a process in accordance with present invention; and

FIG. 4B depicts a flowchart of one embodiment of a process in accordance with the present invention.

### DETAILED DESCRIPTION OF THE INVENTION

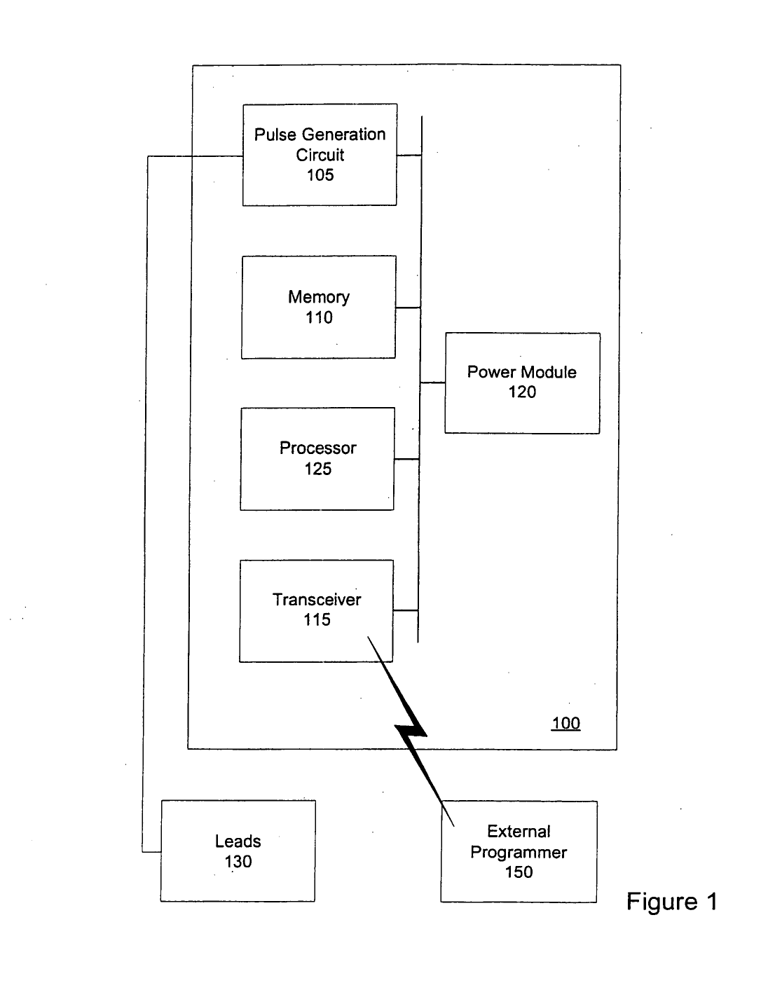

FIGS. 1 through 4B, discussed below, and the various embodiments used to describe the principles of the present invention in this patent document are by way of illustration only and should not be construed in any way to limit the scope of the invention. Those skilled in the art will understand that the principles of the present invention may be implemented in any suitably arranged device. The numerous innovative teachings of the present application will be described with particular reference to the presently preferred embodiment.

In one embodiment of the present invention, there is a system, method, and implantable pulse generator (IPG) device that stores, on the implantable device, two or more stimulus programs, each preferably having one or more stimsets, preferably as prescribed by a doctor or other medical professional. The IPG, whether it is a self-contained implantable pulse generator (SCIPG) or externally-powered implantable pulse generator (EPIPG), communicates with an external patient programmer (EPP) to determine which of the stimulus programs should operate at any given time. An advanced programmer is used to read and write program instructions to the IPG. In this way, the patient is capable of carrying two or more program options within the IPG. If the patient uses an EPIPG, the patient can use any available EPP to power, control and operate the EPIPG. If the patient uses an SCIPG, the patient can use any available EPP to control and operate the SCIPG. In a preferred embodiment, programs stored on the IPG are uploaded to the EPP, and a selected program is validated and retransmitted to the IPG for execution.

The techniques disclosed herein can be used in any implantable medical device, such as a pump, deep-brain stimulation system, etc.

As used herein, an SCIPG is an IPG having an implanted power source, such as a long-lasting or rechargeable battery. An EPIPG is an IPG which receives at least some of its operating power from an external power transmitter, preferably in the form of a RF signal. The external power transmitter, in the preferred embodiment, is built into the external programmer.

FIG. 1 shows a diagram of the components of an IPG 100 in accordance with the preferred embodiment. The implanted device comprises, but is not limited to, a pulse generation circuit 105, a non-volatile memory 110, a transceiver 115, a power module 120, and a processor 125. Memory 110 may also include volatile memory (not shown).

In an SCIPG, the power module 120 will include a long-term battery or a rechargeable battery and a voltage detection and regulation circuit. In an EPIPG, and in an SCIPG with a rechargeable battery, the power module 120 will include a circuit for converting radio-frequency (RF) energy (or other energy) into direct current. In either case, the power module 120 is connected to power the processor 125 and the pulse generation circuit 105.

One example of a conventional SCIPG may be an SCIPG manufactured by Advanced Neuromodulation Systems, Inc. such as the Genesis® system, part number 3608.

The pulse generation circuit 105 is connected to receive power from power module 120 and to be controlled by processor 125. Processor 125 is connected to receive power from power module 120 and to read from, and write to, non-volatile memory 110. Further, processor 125 is connected to receive and decode data from transceiver 115. Note that in different embodiments, transceiver 115 may only be a receiver, while in preferred embodiments, processor 125 is connected to also transmit data via transceiver 115. Further, in various embodiments, transceiver 115 receives power signals for operating or recharging the IPG, transmits, and receives.

Transceiver 115 is positioned to receive RF commands from an external programmer 150, and to deliver these commands to processor 125. Further, in an EPIPG, the receiver 115 is configured to receive RF power signals, and to deliver these to power module 120. The external programmer 150 also includes memory, a processor, transceiver circuitry, and input/output devices and or functionality (such as a display, keyboard, user input buttons, and the like) (not shown).

Non-volatile memory 110 contains programming and control data, and can be written to and read from by processor 125.

Leads 130 are implanted in the patient's epidural space (or other locations), as described above or known to those of skill in the art. Leads 130 connect with pulse generation circuit 105, optionally via lead extensions (not shown).

Leads 130, in one embodiment, have multiple electrodes, each of which can be independently controlled by the pulse generation circuit 105. Each electrode can be individually set as positive (acting as an anode), negative (acting as a cathode), or high impedance (turned off). The pulse generation circuit 105, under control of the processor 125, also controls the pulse amplitude, pulse width, and pulse frequency to each electrode on the leads 130.

Also shown here, although not a part of the IPG 100 itself, is external programmer 150, which communicates with transceiver 115. External programmer 150 can be either an external patient programmer (EPP), which is typically carried and operated by the patient, or an advanced programmer, which is typically operated by the patient's physician or clinician. External programmer 150 will typically communicate with transceiver 115 via an antenna (not shown), placed on or near the patient's body proximal to the IPG 100, via near-field or far-field technology.

In a conventional EPIPG, the external programmer is used to send both a power signal and pulse-generation instructions, on a real-time basis, to the EPIPG. In this case, the programming for the EPIPG is stored on the external programmer.

One of the differences between the preferred embodiment and conventional systems is that, in the preferred embodiment, multiple treatment programs are stored on the IPG by using an advanced programmer by the patient's physician or other professional, then the patient can use his external programmer to select between the multiple programs and/or change customizable options such as multiple pulse amplitude parameters. In the case of an EPIPG, the external programmer will also supply a power signal to the IPG, and in the case of a rechargeable SCIPG, the external programmer will deliver power to recharge the battery.

According to one embodiment, with multiple treatment programs stored on the IPG, the patient can use any compatible external programmer to select between the programs or change options. In this way, unlike in conventional EPIPGs, the patient is not “shackled” to his specific, prescribed external programmer, and can use any available external programmer, such as one at his physician's office, or a spare he might store in his car.

By storing multiple treatment programs, each of which has been prescribed and stored on the IPG by the physician, the patient is able to select the appropriate treatment program for his current activities, using any available compatible external programmer, without having to worry that the programmer will attempt to operate his IPG with a non-prescribed, and potentially harmful, program.

Because, according to one embodiment, all treatment programming is stored on the IPG, the only difference between an SCIPG and an EPIPG, in this case, is whether the power source is also implanted, as in the SCIPG. All treatment programming is stored in the IPG, and both types of IPGs allow the programs to be selected using an external programmer. Since the external programmer is no longer required to be “tied” to a specific patient or IPG, any compatible external programmer can be used, including a “universal” external programmer.

A program consists of one or more stimulation settings, also referred to herein as “stimsets.” The programmed stimulation settings specifically define and characterize the administered electric pulse stimulation. Other information related to stimulation settings, applications, and pain management, not necessary for an understanding of the presently preferred embodiments, is found in U.S. Pat. No. 5,938,690, filed 7 Jun. 1996 and issued 17 Aug. 1999, U.S. Pat. No. 6,609,031, filed 7 Jun. 1996 and issued 19 Aug. 2003, and U.S. patent application Ser. No. 10/120,953, filed 11 Apr. 2002 and published 22 Aug. 2002 as United States Patent Application Publication No. 2002/0116036, all of which are hereby incorporated by reference.

In one embodiment, each stimset is comprised of an electrode configuration and stimulation amplitude, stimulation frequency, and/or stimulation pulse width, and those of skill in the art will recognize that other parameters can be included. The electrode configuration defines whether each electrode is on or off and, if on, the polarity of that electrode. The amplitude is the intensity of the applied electric pulse. The frequency is the number of times the electrodes are turned on each second. The pulse width is the amount of time the electrodes are left on during each cycle.

A program is defined as having at least one stimset, and generally corresponds to providing a treatment relating to a specific part of a patient's body. A program can have multiple stimsets; in this case, each stimset is applied sequentially, repeatedly, and/or randomly. Preferably, each program is applied so that the patient experiences the combined effect of each stimset, as if they were being applied simultaneously.

For example, a first stimset may provide relief to a patient's right leg, and a second stimset may provide relief to a patient's left leg. According to one embodiment, then, there will be at least three programs stored on the patient's IPG:

Program 1 comprises the first stimset;

Program 2 comprises the second stimset; and

Program 3 comprises both the first and second stimsets.

In this case, when the patient uses program 1 on the IPG, she would feel relief in her right leg, program 2 would provide relief in her left leg, and program 3 would provide relief in both legs.

A program comprising more than one stimset is referred to herein as a “multistim program.”

In one embodiment, the IPG is capable of storing up to 24 different programs, each program having up to 8 stimsets. Of course, in other embodiments, the IPG can store a greater number of programs, each having associated a greater number of stimsets, if desired.

In the preferred embodiment, all active electrodes in a stimset receive the same stimulation input, including the same pulse width, pulse frequency, and pulse amplitude. Each electrode in the stimset is assigned a polarity of positive, negative, or off. For example, a first stimset for an 8-electrode lead can be defined as having an amplitude of approximately 4 mA, delivered with a 280 microsecond pulse width and an 80 Hz frequency, with the following electrode polarities, with “+” indicating a positive polarity (anode), “−” indicating a negative polarity (cathode), and “0” indicates that the electrode is off:

Electrode #

| | 0 | 1 | 2 | 3 | 4 | 5 | 6 | 7 |
|---|---|---|---|---|---|---|---|---|
| Polarity | + | + | 0 | 0 | − | − | + | 0 |

Note that in the preferred embodiment, every stimset has at least one anode and one cathode. In an alternate embodiment, the IPG itself (e.g., the case) can act as an anode. A second stimset for an 8-electrode lead can be defined as having an amplitude of approximately at 4.2 mA, delivered with a 240 microsecond pulse width and an 80 Hz frequency, with the following electrode polarities:

Electrode #

| | 0 | 1 | 2 | 3 | 4 | 5 | 6 | 7 |
|---|---|---|---|---|---|---|---|---|
| Polarity | − | − | 0 | + | − | 0 | + | 0 |

Then, if a multistim program contains both the first and second stimset (as the exemplary Program 3, above), as the program is executed, the IPG will rapidly alternate between the first and second stimsets, so that the patient experiences the combined effect of both stimsets in the multistim program. In the currently preferred embodiment, all stimsets in a program have the same frequency, but other embodiments allow for different frequencies in a single program.

A typical pulse, in a preferred embodiment, is approximately 4 mA, delivered with a 280 microsecond pulse width and an 80 Hz frequency. Those of skill in the art will recognize that the pulse amplitude can also be defined in volts.

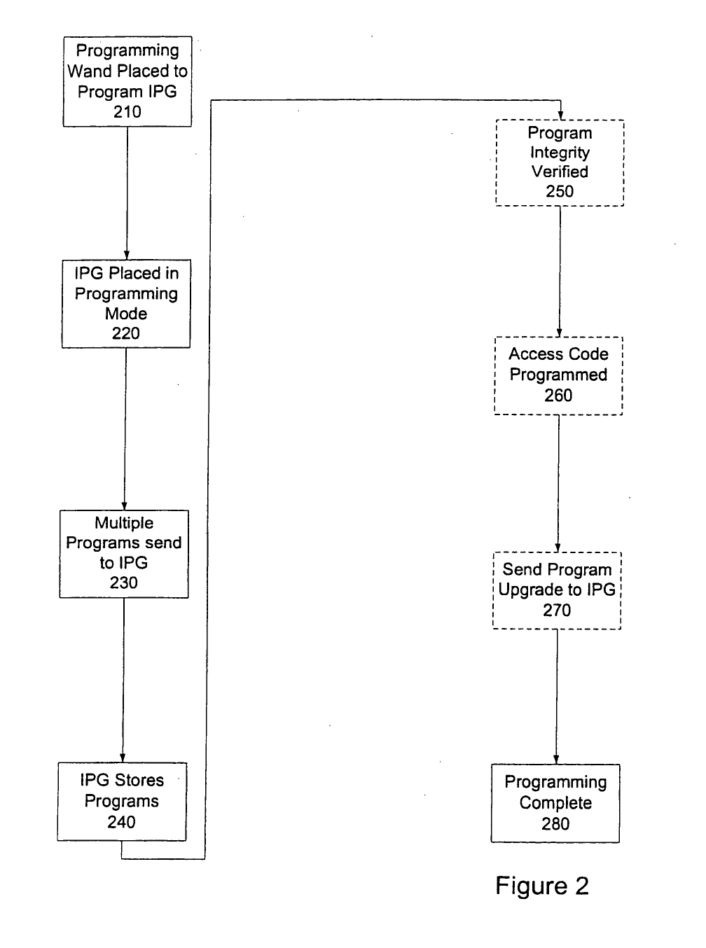

FIG. 2 depicts a flowchart of a process for programming the IPG with multiple treatment-protocol programs. Note that this process is used to program an already-implanted IPG; a similar process can be used to pre-program the IPG before implantation.

This process is typically performed by a physician or other professional using an advanced programmer, as described herein. Generally, this programming process is not one that would normally be performed by a patient, but could be so if the patient were properly trained or under the guidance of a trained professional.

First, a programming wand will be placed in a location proximate to the IPG or the IPG antenna (step 210). In other embodiments, “far-field” programming can be used. Next, preferably using an RF signal, the advanced programmer will place the IPG into programming mode (step 220).

The advanced programmer is then used to send at least two patient-prescribed treatment-protocol programs to the IPG (step 230). The IPG will store these programs in non-volatile memory (step 240).

Optionally, the IPG will then verify correct receipt of the programs using a checksum or other method (step 250), and can receive an access code to restrict access to the treatment protocol programming (step 260). Also, the same programming technique can be used to replace or upgrade the IPG internal programming (step 270).

Programming is then complete (step 280). The IPG is, at this point, programmed with multiple treatment-protocol programs, which can be selected by the patient as described herein.

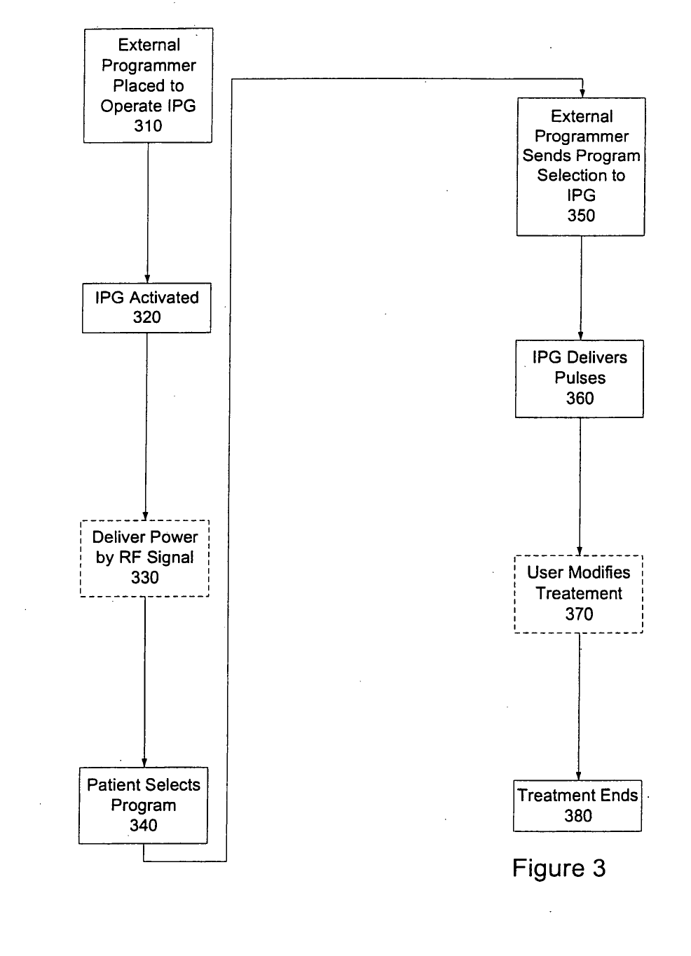

FIG. 3 depicts a flowchart that describes the use of an IPG having multiple treatment-protocol programs stored within. This process is generally performed by the patient.

First, the external programmer will be placed in a location proximate to the IPG or the IPG antenna (step 310). Next, preferably using an RF signal, the external programmer will activate the IPG (step 320).

During operation, the external programmer will optionally, as in the case of an EPIPG, supply power to the IPG, preferably using an RF signal (step 330). The patient will select (via user input) the treatment protocol on the external programmer (step 340), and the external programmer will send an RF signal to the IPG to indicate the selected treatment-protocol program (step 350). Alternately, if a treatment protocol selection is not sent by the external programmer, the IPG will select one of the stored treatment-protocol programs as the “default” program.

The IPG delivers the pulse stimulus, as described herein, according to the selected treatment-protocol program (step 360) and its associated stimsets. Optionally, the user can modify the intensity or other aspects of the treatment as needed, using the external programmer (step 370). For example, a typical modification is to change the intensity setting using the external programmer, causing the IPG to adjust the pulse amplitude delivered to the lead electrodes.

When the treatment program is completed, or when the user chooses, the pulse-stimulus treatment ends (step 380).

In accordance with the present invention, the treatment-protocol program(s) stored within the IPG 100 may be uploaded to the external programmer or other external device 150. The uploading of the treatment-protocol program(s) to the external device 150 advantageously provides the ability for review, validation, and/or modification of the programs, and for programming of the IPG 100. As used herein, the term “program” may also refer to code or “program information” which may be other information describing the program, program name, parameters and/or settings of the program, such as abbreviated program and/or stimset information, and the like.

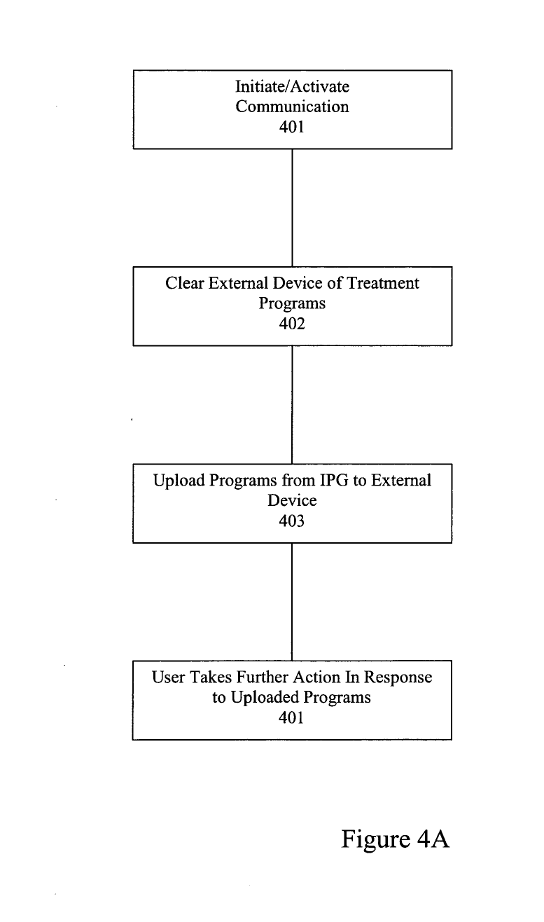

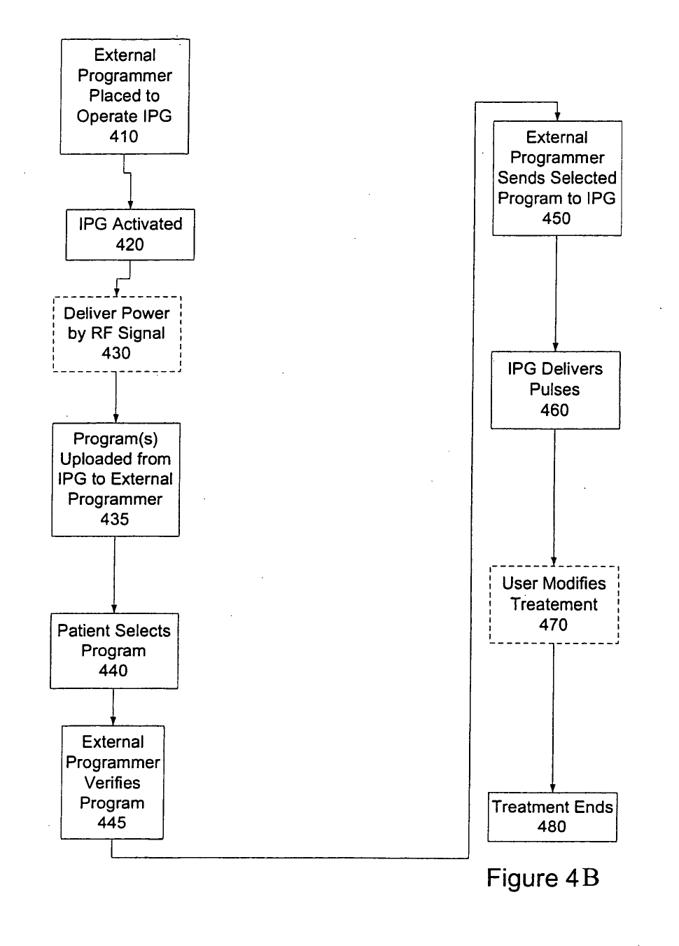

FIG. 4A illustrates the overall process 400 of the present invention. A communication session is initiated (i.e., the IPG is activated) by the external device 150 (step 401). At a step 402, the memory (not shown) of the external device 150 is cleared of all treatment programs. Upon request by the external device 150, the program(s) stored in the IPG 100 are uploaded to the external device 150 (step 403). Alternatively, this information can be uploaded automatically upon establishment of the communication session or activation of the IPG 100. Once uploaded, the user (patient, medical professional, etc.) may utilize the program or program information as desired, such as to review, validate, select, or modify the programs stored for further action (step 404). In one embodiment, the step 404 may include all of the steps described in FIG. 3, or a portion of such process (e.g., steps 340 thru 380). The uploaded programs may be reviewed via an I/O device, such as a display (not shown) of the external device 150. Additional I/O device(s) of the external device 150, such as a keyboard or user input/control buttons, and the like (not shown) may be utilized by the user to take or perform further action(s).

The clearing (or deleting/erasing) of the contents of memory (within the external device 150) occurs prior to, or substantially contemporaneous with, the uploading and storing of the treatment programs from the IPG 100. As such, clearing (or deleting) all previously stored treatment programs in the external device could take place (1) in response to a user request (at any time, even before a communication session is established with the IPG 100), (2) upon initiation/activation of the communication session with the IPG (step 401), or (3) upon the uploading of treatment programs from the IPG 100 (memory may be overwritten), or using some other method and at some other point in time during the process before storing the new program information. Method (2) or (3) could further include a user prompt generated by the external device 150 (to the user) to confirm deletion of previously stored programs. User prompts could also be used with any other steps in the process. As such, the term “clearing” as used herein, refers to the erasure or deletion of the contents of memory, either by erasing, deleting, or overwriting the contents with other contents.

It may be useful for the external device 150 to allocate a predetermined portion of memory to store the treatment programs uploaded from the IPG 100. This may provide a more efficient and safer way to delete previously stored treatment programs in preparation to receiving programs from the IPG 100. This would allow for all memory allocated for storing uploaded treatment programs to be cleared, regardless of the number of treatment programs that might be stored. Of course, the external device 150 may be configured to overwrite the existing memory contents with the newly uploaded treatment programs.

Thus, the memory of the external device (memory that may be storing any previously-stored treatment programs) is cleared in response to a user request (specific request from user, or as part of the overall process of uploading treatment programs from the IPG 100 to the external device 150). One benefit of the present invention is that an external device 150 may be used with different implants—as the external device programmer or controller 150 clears its memory of all prior or “old” treatment programs and downloads those treatment programs applicable to (or authorized by) the current IPG 100. Thus, the external device 150 gets “loaded” with those programs as determined by the configuration of the IPG 100 (i.e., those programs stored within the IPG 100).

FIG. 4B depicts a flowchart that describes one embodiment of an IPG 100 having multiple treatment-protocol programs stored within, in which the program(s) are uploaded from the IPG 100 to the external device 150 for execution. This process is generally initiated by the patient or other user.

First, the external device 150 is placed in a location proximate to the IPG 100 or the IPG antenna (step 410) and, using an activation signal, the external device 150 activates the IPG for a communication session (step 420). This may be done using far-field or near-field communication signal(s). In the case of an EPIPG, the external device 150 may also supply power to the IPG 100 (using an RF signal) (step 430).

Memory of the external device 150 storing any previously-stored or “old” treatment programs is cleared. In a preferred embodiment, clearing the external device memory of all previously-stored treatment programs occurs prior to, or contemporaneously (overwriting the memory) with, the uploading of treatment programs from the IPG 100. One or more treatment protocol programs (or program information) is transmitted (or uploaded) from the IPG 100 to the external device 150, and stored in the external device 150 (step 435). The uploaded program(s) may be displayed on display device (not shown) of the external device 150 or other device. A user (usually the patient) selects, via input means, (step 410) one of the uploaded treatment protocol programs on the external device 150 that have been uploaded or received from the programs stored in the IPG 100.

The external device 150 optionally validates the selected treatment protocol program to verify that the stimset(s) in the selected program are within a correct effective range for the patient, without being either ineffective or uncomfortable for the patient (step 445). When necessary or desired, the external device 150 can modify the selected program (or other uploaded programs) as appropriate for the patient, either automatically or through user input.

The external device 150 then transmits the selected treatment protocol program to the IPG 100 for storage in an active memory of the IPG 100 for execution by the IPG (step 450). Alternatively, one embodiment may include the execution of an IPG-stored program according to an instruction received from the external device 150, i.e., no program is re-downloaded to the IPG 100 from the external device 150, and thus only information instructing the IPG 100 to execute one of the IPG-stored programs is transmitted to the IPG 100 (e.g., only information identifying the selected program, such as Program No. 3 of 24, is transmitted). In other embodiments, the program (or stimset(s)) is not directly transferred from the program memory to the active memory within the IPG 100 to enable execution. Instead, the programs are uploaded to the external device 150, a program is selected, and the selected program is transferred back to the active memory (e.g., the executable memory) of the IPG 100 for execution of the program. Additionally, these embodiments may include the real-time receipt of pulse-generation signals from the external programmer or device 150, where the selected program is executed by the external programmer or device 150.

The IPG 100 generates and delivers the pulse stimulus, as described herein, according to the treatment protocol program selected (i.e, the program downloaded to the IPG 100, or the program selected to be executed by the instructions downloaded to the IPG 100) (step 460).

Optionally, the user may modify the intensity or other aspects of the selected treatment program which are allowed to be modified by the user, as needed, using the external programmer or device 150 (step 470). For example, a typical modification is to change the intensity setting using the external programmer device 150, causing the IPG 100 to adjust the pulse amplitude delivered to the lead electrodes.

When the treatment program is completed, or when the user chooses, the pulse-stimulus treatment ends (step 480).

According to some embodiments, both the external programmer or device 150 and the IPG 100 have unique serial numbers. These serial numbers are exchanged between the IPG 100 and external device 150, and both the IPG 150 and external device 150 store the serial number of the device they have last communicated with. Further, the external programmer 150 may cache any programs that have been uploaded from an IPG 100. In at least some of these embodiments, when the programs are to be uploaded as in step 435 above, if the corresponding stored serial numbers indicate that these particular IPG 100 and external programmer devices 150 last communicated with each other, then the external programmer device 150 may utilize the cached programs, having verified that they were received from this specific IPG 100, instead of re-uploading them from the IPG 100. This process provides an optimization of the process when the same IPG/external programmer is reused, without “tying” the IPG to a specific external programmer as in known systems.

In another embodiment, the IPG 100 includes a radio-frequency identification (RFID) tag, known to those of skill in the art.

Those skilled in the art will recognize that, for simplicity and clarity, the full structure and operation of all devices and processes suitable for use with the present invention is not being depicted or described herein. Instead, only so much of an implantable pulse generator and supporting hardware as is unique to the present invention or necessary for an understanding of the present invention is depicted and described. The remainder of the construction and operation of the IPGs described herein may conform to any of the various current implementations and practices known in the art.

Those of skill in the art will also recognize that not all steps in the above-described processes must be performed in the order described. Further, not all steps of any process, particularly the optional steps, must necessarily be performed in conjunction with all other steps, and can be omitted from the process or performed independent of other steps of the process.

It is important to note that while the present invention has been described in the context of a fully functional system, those skilled in the art will appreciate that at least portions of the mechanism of the present invention are capable of being distributed in the form of an instruction set contained within a machine usable medium in any of a variety of forms, and that the present invention applies equally regardless of the particular type of instruction or signal bearing medium utilized to actually carry out the distribution. Examples of machine usable mediums include: nonvolatile, hard-coded type mediums such as read only memories (ROMs) or erasable, electrically programmable read only memories (EEPROMs), user-recordable type mediums such as floppy disks, hard disk drives and compact disk read only memories (CD-ROMs) or digital versatile disks (DVDs), and transmission type mediums such as digital and analog communication links.

Although an exemplary embodiment of the present invention has been described in detail, those skilled in the art will understand that various changes, substitutions, variations, and improvements of the invention disclosed herein may be made without departing from the spirit and scope of the invention in its broadest form.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC §112 unless the exact words “means for” are followed by a participle.

It may be advantageous to set forth definitions of certain words or phrases used throughout this patent document: the terms “include” and “comprise,” as well as derivatives thereof, mean inclusion without limitation; the term “or” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and if the term “controller” is utilized herein, it means any device, system or part thereof that controls at least one operation, whether such a device is implemented in hardware, firmware, software or some combination of at least two of the same. It should be noted that the functionality associated with any particular controller may be centralized or distributed, whether locally or remotely.

What is claimed is:

1. An implantable device, comprising:
a processor operable for controlling operation of the implantable device;
a memory coupled to the processor, the memory operable for storing a treatment program; and
a communication circuit operable for transmitting the stored treatment program to an external device.

2. The device in accordance with claim 1 wherein the stored treatment program was previously downloaded from another device and stored in the memory of the implantable device.

3. The device in accordance with claim 1 wherein the memory is operable to store at least two treatment programs, and at least one of the treatment programs includes a plurality of stimulation settings.

4. The device in accordance with claim 3 wherein the communication circuit is operable for transmitting the at least two treatment programs to the external device and the stored treatment-protocol programs were previously downloaded from another device and stored in the memory of the implantable device.

5. A method of controlling operation of an implantable device, comprising:
storing within a memory in the implantable device a treatment program, the treatment program operable for controlling the implantable device;
transmitting the stored treatment program from the implantable device to the external device;
receiving from the external device a treatment program; and
controlling operation of the implantable device in response to the received treatment program.

6. The method in accordance with claim 5 further comprising:
clearing memory within the external device of previously stored treatment programs, the memory operable for storing treatment programs.

7. The method in accordance with claim 5 wherein the received treatment program is substantially the same as the stored treatment program previously transmitted to the external device.

8. The method in accordance with claim 5 further comprising:
validating the treatment program transmitted from the implantable device to the external device and thereafter transmitting the validated treatment program to the implantable device.

9. The method in accordance with claim 5 wherein storing the treatment program comprises storing a plurality of treatment programs, and transmitting the stored treatment program comprises transmitting the plurality of stored treatment programs, and the method further comprises:
receiving from the external device a selected treatment program.

10. The method in accordance with claim 9 wherein the received selected treatment program is substantially the same as one of the plurality of stored treatment programs previously transmitted to the external device.

11. The method in accordance with claim 9 wherein at least one of the plurality of treatment programs includes a plurality of stimulation settings.

12. The method in accordance with claim 5 further comprising:
storing within the memory in the implanted device a plurality of treatment programs, the plurality of treatment programs each operable for controlling the implantable device;
transmitting the plurality of stored treatment programs from the implantable device to an external device;
receiving at the external device the plurality of transmitted treatment programs;
selecting through the external device a one of the plurality of treatment programs received by the external device;
transmitting the selected treatment program from the external device to the implanted device; and
receiving at the implanted device the selected treatment program.

13. The method in accordance with claim 12 further comprising:
clearing memory within the external device of previously stored treatment programs, the memory operable for storing treatment programs.

14. The method in accordance with claim 12 wherein at least two treatment programs are stored, and at least one of the treatment programs includes a plurality of stimulation settings.

15. An external device operable for monitoring one or more treatment programs stored within an implantable device, comprising:
a communication circuit operable for wirelessly receiving from the implantable device the one or more treatment programs stored within the implantable device; and
a display means operable for displaying information related to the one or more treatment programs to a user; and
an input means operable for selecting one of the one or more of the received treatment programs to control operation of the implantable device; and
wherein the communication circuit is further operable for transmitting the selected treatment program to the implantable device, the transmitted selected treatment program operable for controlling operation of the implantable device.

16. The device in accordance with claim 15 further comprising:
memory operable for storing treatment programs; and
means for clearing the memory, the memory operable for storing the one or more received treatment programs from the implantable device.

17. The device in accordance with claim 15 wherein the received treatment programs were previously downloaded from another device and stored in the memory of the implantable device.

18. The device in accordance with claim 15 wherein the one or more treatment programs comprises at least two treatment programs, and at least one of the treatment programs includes a plurality of stimulation settings.
